(19) United States
(12) Patent Application Publication
(10) Pub. No.: US 2010/0016409 A1
(43) Pub. Date: Jan. 21, 2010

(54) Rna Nanoparticles and Nanotubes

(75) Inventors: Yaroslava G. Yingling, Cary, NC (US); Bruce A. Shapiro, Gaithersburg, MD (US)

Correspondence Address: EDWARDS ANGELL PALMER & DODGE LLP, PO BOX 55874, BOSTON, MA, 02205, US

(73) Assignee: Government of the US, as represented by the Secretary, Department of Health and Human Services, Rockville, MD (US)

(21) Appl. No.: 12/227,955

(22) PCT Filed: May 31, 2007
(86) PCT No.: PCT/US2007/013027

§ 371 Date: Sep. 1, 2009

Related U.S. Application Data
(60) Provisional application No. 60/810,283, filed on Jun. 2, 2006.
(60) Provisional application No. 60/918,181, filed on Mar. 14, 2007.

(51) Int. Cl. A61K 31/7088; C07H 21/02; A61P 35/00; A61P 31/22; A61P 25/28; A61P 3/04

(52) U.S. Cl. 514/44 R; 536/23.1; 977/773

(57) ABSTRACT

The instant invention provides polyvalent RNA nanoparticles comprising RNA motifs as building blocks that can form RNA nanotubes. The polyvalent RNA nanoparticles are suitable for therapeutic or diagnostic use in a number of diseases or disorders.

### CROSS-REFERENCE TO RELATED APPLICATION

This application claims the benefit of U.S. Provisional Application No. 60/810,283 which was filed on Jun. 2, 2006, and U.S. Provisional Application No. 60/918,181 which was filed on Mar. 14, 2007, the entire disclosures of which are hereby incorporated in their entireties.

### GOVERNMENT FUNDING

Research supporting this application was carried out by the United States of America as represented by the Secretary, Department of Health and Human Services.

### BACKGROUND OF THE INVENTION

The rapidly expanding field of nanobiology opens up the possibilities for the development of new methods and compositions that can be used for the diagnosis, prognosis, and treatment of various diseases such as cancer. While an increasing number of novel drugs and therapeutic agents are being discovered, the problem of delivering them specifically to the desired site or cell has not been solved. Nanoparticles are ideal drug delivery devices due to their novel properties and functions and ability to operate at the same scale as biological entities. Nanoparticles, because of their small size, can penetrate through smaller capillaries and are taken up by cells, which allow efficient drug accumulation at the target sites (Panyam J et al., Fluorescence and electron microscopy probes for cellular and tissue uptake of poly (D, L-lactide-co-glycolide) nanoparticles, Int J Pharm. 262:1-11, 2003). There are several issues that are crucial for efficient design and drug delivery by nanoparticles, including the efficient attachment of drugs and vectors, controlled drug release, size, toxicity, biodegradability, and activity of the nanoparticle. Moreover, for successful design one needs to understand and control the intermolecular associations, based on natural favorability of interactions and various physical components.

Targeted delivery of nanoparticles can be achieved by either passive or active targeting. Active targeting of a therapeutic agent is achieved by conjugating the therapeutic agent or the carrier system to a tissue or cell-specific ligand (Lamprecht et al., Biodegradable nanoparticles for targeted drug delivery in treatment of inflammatory bowel disease, J Pharmacol Exp Ther. 299:775-81, 2002). Passive targeting is achieved by coupling the therapeutic agent to a macromolecule that passively reaches the target organ (Monsky W L et al., Augmentation of transvascular transport of macromolecules and nanoparticles in tumors using vascular endothelial growth factor, Cancer Res. 59:4129-35, 1999). Drugs encapsulated in nanoparticles or drugs coupled to macromolecules such as high molecular weight polymers passively target tumor tissue through the enhanced permeation and retention effect (Maeda H, The enhanced permeability and retention (EPR) effect in tumor vasculature: the key role of tumor-selective macromolecular drug targeting, Adv Enzyme Regul. 41:189-207, 2001; Sahoo S K et al., Pegylated zinc protoporphyrin: a water-soluble heme oxygenase inhibitor with tumor-targeting capacity, Bioconjugate Chem. 13:1031-8, 2002).

As macromolecules such as proteins and nucleic acids play a larger role in the therapy of disease and traditional pharmaceutical small molecules are abandoned during development due to their inability to effectively reach their intended target, improved drug delivery systems are needed. The delivery of a wide variety of drugs is hindered because they have difficulty crossing the blood brain barrier. A characteristic function of nanoparticles is their ability to deliver drugs across biological barriers to the target site and to protect the drugs from the biological environment until they reach the target site. Earlier detection and treatment of disease would provide significant benefit to the patient; however the development of effective methodologies that allow for early detection and treatment remain to be realized. Ribonucleic acid (RNA) nanotechnology provides a new approach for the treatment and detection of disease, and is one of the most promising applications for overcoming disease. The goal of nanotechnology-based RNA therapy is to provide a safe, efficient and specific nonpathogenic nanodevice for the delivery of multiple therapeutic RNAs, including siRNA, aptamers, ribozymes, antisense RNA, molecular beacons, and sensors. For example, small interfering RNA (siRNA) can down-regulate genes responsible for cancer, viral pathogenesis, and inflammatory conditions (Yano, J et al. Clin Cancer Res 2004, 10, (22), 7721-6; Flynn, M A et al. J Inflamm (Lond) 2004, 1, (1), 4) and, thus, is very important for target based drug discovery and development. Synthetic siRNA can be added to cells in order to artificially induce RNA interference and to silence the expression of target genes in a variety of organisms and cell types. siRNA offer enormous potential as a therapeutic agent with higher specifity, more effectiveness and less toxic effect than traditional drugs. However, the successful application of siRNA therapeutics depends on efficient delivery to the appropriate cell. Nanoparticles can be used as delivery vehicles for siRNA therapeutics. The minimum requirements for such nanoparticles are that they be less than 100 nm in diameter to penetrate cell membranes, have negligible toxicity, and have protection from exonuclease degradation.

Although protein nanoparticles are widely used and researched for therapeutic and diagnostic purposes, protein nanoparticles have been shown to elicit an immune response in animals that makes them difficult to use for long-term administration. Conversely, protein free RNAs elicit only a low level immune response, and therefore, are more practical for long-term administration. Thus all-RNA nanoparticles can be used in long-term treatment of chronic diseases such as cancer, hepatitis B, or AIDS. RNA is immune to nuclease degradation. A limiting factor for RNA nanoparticles is their stability in the bloodstream and nonspecific cellular uptake, posing a requirement of high dosage. The relative stability can be controlled by the chemical modification of the backbone, addition of proteins, lipids (Yano et al., as above), self-contained compact structures or polymeric chains. (Schiffelers, R M et al. Nucleic Acids Res 2004, 32, (19), e149; Howard, K A et al. J. Mol Ther. 2006).

Accordingly, a safe and efficient nanoparticle needs to be designed for the delivery of effective therapeutic and diagnostic RNAs.

### SUMMARY OF THE INVENTION

As described below, the present invention provides RNA nanoparticles that provide a number of improvements over nanoparticles currently available. Moreover, the instant invention provides methods that allow for the design of RNA nanoparticles as described herein.

Accordingly, in at least one aspect the invention provides a polyvalent RNA nanoparticle comprising RNA motifs as building blocks.

In one embodiment, the building blocks comprise a motif that allows for non-covalent assembly between 2 or more building blocks.

In a particular embodiment, the RNA motifs are RNA I or RNA II motifs. In another particular embodiment, the RNA motifs are RNA I and RNA II motifs. In another particular embodiment, the motifs are kissing loops. In still another embodiment, the RNA motifs are RNA I inverse (RNA Ii) or RNA II inverse (RNA IIi) motifs. In a related embodiment, the RNA motifs are RNA I inverse (RNA Ii) and RNA II inverse (RNA IIi) motifs.

In another embodiment of the aspect, the polyvalent RNA nanoparticle is in the shape of a ring. In one embodiment, the ring comprises one or more building blocks. In another embodiment, the ring comprises 6 building blocks that form a hexameric ring. In another embodiment, there is a 120 degree angle at the loop-loop interactions at the corners of the hexameric ring.

In another embodiment, the polyvalent RNA nanoparticle is in the shape of a square. As a non-limiting example, the square can be formed using a right angle non-covalently linked motif. In a particular embodiment, the square comprises one or more building blocks.

In another embodiment, the polyvalent RNA nanoparticle is in the shape of a triangle. As a non-limiting example, the triangle can be formed using a 60 degree non-covalently linked motif. In a particular embodiment, the triangle comprises one or more building blocks.

In another particular embodiment, the polyvalent RNA nanoparticle of any of the above aspects comprises both RNA Ii and RNA IIi building blocks.

In still another embodiment of the polyvalent RNA nanoparticle of any of the above aspects, the building blocks are held together by non-covalent loop-loop contacts. In a further particular embodiment, each building block contains two or more hairpin loops. In another particular embodiment, the hairpin loops are connected by a helix.

In another embodiment of any one of the above aspects, the building blocks have 5′ or 3′ sticky ends which are denoted by a single stranded motif.

In a particular embodiment, the 5′ or 3′ sticky ends are located in the middle of a helix. In another particular embodiment, the 5′ and 3′ sticky ends are engineered as sticky ends for self-assembly of nanorings into an RNA nanotube. In still a further embodiment, the polyvalent RNA nanoparticle is capable of self-assembly into a nanotube. In another particular embodiment, the self-assembly can occur as a single-step process. In still another particular embodiment, the self-assembly can occur as a hierarchical process.

In another particular embodiment, the RNA nanoparticles are connected via complementary sticky ends.

In another embodiment, the 5′ and 3′ sticky ends are positions for conjugation of one or more therapeutic, diagnostic, or delivery agents. In a further embodiment, the one or more therapeutic, diagnostic, or delivery agents are directly included in the building block sequences. In still another embodiment of the aspect, therapeutic agent is selected from the group consisting of: siRNA, aptamers, oligonucleotides, antisense RNA, chemotherapeutic agents, ribozymes, and, fluorescent beads, heavy metals, radioisotopes, quantum dots, and molecular beacons. In a particular embodiment, the delivery agent is selected from the group consisting of: liposomes, antibodies, polyamines, and polyglycols, e.g., polyethylene glycol. In another particular embodiment, the delivery agent can be RNA by itself.

In a particular embodiment, the polyvalent RNA nanoparticle or the polyvalent RNA nanotube of any of the above mentioned aspects is used in, for example, drug delivery, imaging, nanocircuits, cell growth surfaces, medical implants, medical testing, or gene therapy.

In another embodiment, a drug delivery composition comprises the polyvalent RNA nanoparticle of the above-mentioned aspects, where the drug delivery composition can gain entry in to a cell or tissue.

In a particular embodiment, the drug delivery composition further comprises a second therapeutic agent. In a further embodiment, the second therapeutic agent is selected from the group consisting of chemotherapeutic agents, cardiovascular drugs, respiratory drugs, sympathomimetic drugs, cholinomimetic drugs, adrenergic or adrenergic neuron blocking drugs, analgesics/antipyretics, anesthetics, antiasthmatics, antibiotics, antidepressants, antidiabetics, antifungals, antihypertensives, anti-inflammatories, antianxiety agents, immunosuppressive agents, immunomodulatory agents, antimigraine agents, sedatives/hypnotics, antianginal agents, antipsychotics, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterials, antivirals, antimicrobials, anti-infectives, bronchodialators, hormones, hypoglycemic agents, hypolipidemic agents, proteins, peptides, nucleic acids, agents useful for erythropoiesis stimulation, antiulcer/antireflux agents, antinauseants/antiemetics and oil-soluble vitamins, or combinations thereof.

In another further embodiment, the chemotherapeutic agent is selected from the group consisting of acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin;.enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfomithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amnifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Additional cancer therapeutics include monoclonal antibodies such as rituximab, trastuzumab and cetuximab.

In another embodiement, the invention features a method of treating or preventing a disease or disorder, the method comprising administering to a subject in need thereof an effective amount of a polyvalent RNA nanoparticle or nanotube of any one of the above aspects.

In a particular embodiment, the disease or disorder is selected from the group consisting of: Adenoma, Ageing, AIDS, Alopecia, Alzheimer's disease, Anemia, Arthritis, Asthma, Atherosclerosis, Cancer, Cardiac conditions or disease, Diabetes mellitus, Foodborne illness, Hemophilia A-E, Herpes, Huntington's disease, Hypertension, Headache, Influenza, Multiple Sclerosis, Myasthenia gravis, Neoplasm, Obesity, Osteoarthritis, Pancreatitis, Parkinson's disease, Pelvic inflammatory disease, Peritonitis, Periodontal disease, Rheumatoid arthritis, Sepsis, Sickle-cell disease, Teratoma, Ulcerative colitis, Uveitis.

In another aspect, the invention features a method for making the polyvalent nanoparticle of any one the above-described aspects, the method comprising overexpressing an RNA sequence comprising an RNA motif in a cell thereby, allowing the RNA sequences to assemble into a polyvalent nanoparticle, thereby making a polyvalent nanoparticle.

In one embodiment, the RNA motifs are RNA I or RNA II motifs. In another embodiment, the motifs are kissing loops. In another particular embodiment, the RNA motifs are RNA I inverse (RNA Ii) or RNA II inverse (RNA IIi) motifs. In yet another embodiment, the RNA motifs are RNA I inverse (RNA Ii) and RNA II inverse (RNA IIi) motifs.

In a further embodiment, the RNAIi sequence comprises cggaugguucg (SEQ ID NO: 1) and the RNAIIi sequence comprises RNAIIi comprises cgaaccauccg (SEQ ID NO: 2).

In another preferred embodiment, the RNAIi and RNAIIi sequences are at opposite ends of a RNA nano-ring building block. In a further embodiment, the RNA nanoring building block comprises GcgcUAUCCAG gggaacggaugguucguuccc UUGGGU Agcgc CCCGUUCUCAA ccgcaccgaaccauccggugcgg UUGGGAACGGG aa CUCUCUCUCUAGAGAGAGAG (SEQ ID NO: 3).

In another embodiment, the nanoparticle is at least 25 nm in size.

### BRIEF DESCRIPTION OF THE DRAWINGS

FIG. 1(a-c) shows the structure of RNA I/RNA II inverse motifs. (a) shows secondary structure and (b) shows tertiary structure. (c) shows temporal angle fluctuations between RNA Ii and RNA IIi stems measured during explicit solvent molecular dynamics simulations.

FIG. 2(a-c) is a 2D diagram showing RNA sequences that form helices capped with the two loops that are based on the RNA I inhibitory and RNA II inhibitory sequences. Free energy was determined using the MFOLD program. The letters highlighted with arrows indicate changes made to preclude other conformational folds.

FIG. 3(a-c) shows the design of an RNA nanoring from two building blocks. (a) shows the secondary structure and (b) and (c) show the three-dimensional structure after MD implicit solvent simulations; (b) shows the face view and (c) shows a view from the side.

FIG. 4(a-d) shows the design of the RNA nanotube. (a) shows the secondary structure and (b) shows the three-dimensional structure of the nanoring including extensions of the 3′ ends of each building block. (c) and (d) show the assembly of nanorings into a nanotube via complementary 3′ ends. (c) is a view from the side and (d) is a view from the top. The rings have identical sequences.

FIG. 5 shows the secondary structure of the RNA nano-ring building block. The structure was folded via the secondary structure prediction program MFOLD.

### DETAILED DESCRIPTION OF THE INVENTION

The instant invention is based on the discovery of multifunctional-engineered nanoparticles. The instant invention provides polyvalent RNA nanoparticles comprising RNA motifs as building blocks that can form RNA nanotubes. The polyvalent RNA nanoparticles are suitable for therapeutic or diagnostic use in a number of diseases or disorders. Specifically, the instant invention provides RNA nanostructures comprising RNA Ii-like or RNA IIi-like that have multiple positions available for conjugation of, for example, therapeutic agents or diagnostic agents, such as imaging agents.

Advantageously, the nanoparticles of the instant invention provide a number of improvements over nanoparticles currently available. For example, the RNA nanoparticles of the invention may not induce a significant immune response like the protein nanoparticles currently used. Moreover, the nanoparticles of the invention are smaller than many currently available nanoparticles and therefore allow for increased efficiency of administration. The nanoparticles described herein comprise multiple RNA subunits each of which has the ability to bind, for example, a therapeutic or diagnostic agent. Moreover, multiple different agents can be present within a single nanoparticle. In an exemplary embodiment, the RNA nanoparticle comprises one or more agents that will specifically target the nanoparticle to a particular type of cell and one or more therapeutic agents. A specific example of this type of nanoparticle would have an agent that specifically targets a particular type of cancer cell one or more cancer therapeutic agents. Previous studies have shown that RNA nanostructures are effective drug delivery vehicles (see, for example, Khaled et al. (2005) Nano Letters 5:1797-1808).

The nanoparticles of the instant invention provide novel drug delivery compositions that, in at least one embodiment, are capable of transport across biological barriers. Moreover, the nanoparticles of the instant invention may not elicit a significant immune response, or elicit only a low-level immune response and are therefore advantageous for administration to subjects.

The RNA nanoparticles described herein have the ability to assemble, e.g., self-assemble, in to higher order structures, e.g.,. a ring or a nanotube. Exemplary RNA nanoparticles that have the ability to self-assemble into nanotubes are hexomeric RNA nanoparticles comprising RNA Ii and/or RNA IIi motifs. RNA nanotubes have use in, for example, nanocircuits, medical implants, gene therapy, scaffolds and medical testing.

### Definitions

The instant invention provides polyvalent RNA nanoparticles comprising RNA motifs as building blocks. The polyvalent RNA nanoparticles described herein can further comprise therapeutic, diagnostic and/or delivery agents. Further, the polyvalent RNA nanoparticles described herein can be used as drug delivery compositions to treat various diseases or conditions.

The following definitions will be useful in understanding the instant invention.

As used herein, the term “comprising” is intended to mean that the compositions and methods include the recited elements, but do not exclude other elements. “Consisting essentially of”, when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

As used herein, the term “administering” is meant to refer to a means of providing the composition to the subject in a manner that results in the composition being inside the subject's body. Such an administration can be by any route including, without limitation, subcutaneous, intradermal, intravenous, intra-arterial, intraperitoneal, and intramuscular.

As used herein, the term “chemotherapeutic agent” is meant to include a compound or molecule that can be used to treat or prevent a cancer. A “chemotherapeutic agent” is meant to include acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlomaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicanycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfomithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis. regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives, balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-i receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonerrnin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thyrnotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

As used herein, the term “effective amount” refers to that amount of a therapeutic agent alone that produces the desired effect (such as treatment of a medical condition such as a disease or the like, or alleviation of a symptom such as pain) in a patient. In some aspects, the phrase refers to an amount of therapeutic agent that, when incorporated into a composition of the invention, provides a preventative effect sufficient to prevent or protect an individual from future medical risk associated with a particular disease or disorder. A physician or veterinarian of ordinary skill can readily determine and prescribe the effective amount of the bioactive agent required to treat and/or prevent the progress of the condition.

As used herein, the term “cancer” is used to mean a condition in which a cell in a subject's body undergoes abnormal, uncontrolled proliferation. Thus, “cancer” is a cell-proliferative disorder. Examples of cancers include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases. The terms “cancer,” “neoplasm,” and “tumor,” are used interchangeably and in either the singular or plural form, refer to cells that have undergone a malignant transformation that makes them pathological to the host organism.

As used herein, a “composition” refers to the combination of an active agent (e.g., a polyvalent RNA nanoparticle). The composition additionally can comprise a pharmaceutically acceptable carrier or excipient and/or one or more therapeutic agents for use in vitro or in vivo.

As used herein, the term “hairpin loop” is meant to refer to a feature of ribonucleic acid (RNA) secondary structure. A hairpin loop occurs when RNA folds back on itself. Base pairing along the double-stranded stems may be either perfectly complementary or may contain mismatches.

As used herein, the term “kissing loop” is meant to refer to the base-pairing formed by complementary sequences in the apical loops of two hairpins which is a basic type of RNA tertiary contact. The simplest kissing interaction is formed between a pair of hairpins each with a GACG tetraloop.

As used herein, the term “nanoparticle” is meant to refer to a particle between 10 nm and 200 nm in size. A nanoparticle according to the invention comprises a ribonucleic acid (RNA). The RNA can be obtained from any source, for example bacteriophages phi 29, HIV, Drosophila, the ribosome, or be a synthetic RNA.

As used herein, the term “nanotube” is meant to refer to the assembly of nanoparticles from RNA into a two or three dimensional structure. The assembly of nano-particles in to nanotubes can be by a process of self-assembly. Self-assembly can occur by ligation, chemical conjugation, covalent linkage, and non-covalent interactions of RNA, especially in the formation of RNA multimeric complexes.

As used herein, the term “motif” in reference to a nanoparticle is meant to refer to a double-stranded or single-stranded ribonucleic acid or analog thereof. Individual motifs are joined together into larger particles by attachment to each other. Attachment can occur by non-covalent linking.

The term “oligonucleotide” as used herein includes linear oligomers of nucleotides or analogs thereof, including deoxyribonucleosides, ribonucleosides, and the like. Typically, oligonucleotides range in size from a few monomeric units, e.g., 3-4, to several hundreds of monomeric units. Olgionucleotides can have inhibitory activity or stimulatory activity.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin Remington's Pharm. Sci., 15th Ed. (Mack Publ. Co., Easton (1975)).

The term “subject” is intended to include organisms needing treatment. Examples of subjects include mammals, e.g., humans, dogs, cows, horses, pigs, sheep, goats, cats, mice, rabbits, rats, and transgenic non-human animals. In certain embodiments, the subject is a human.

As used herein, the term “therapeutic agent” includes a drug and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes. Pharmaceutically active agents include the herein disclosed categories and specific examples. It is not intended that the category be limited by the specific examples. Those of ordinary skill in the art will recognize also numerous other compounds that fall within the categories and that are useful according to the invention. Examples include a growth factor, e.g., NGF or GNDF, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaccutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetominophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

As used herein, the term “treated,” “treating” or “treatment” includes the diminishment or alleviation of at least one symptom associated or caused by the state, disorder or disease being treated. A subject that has been treated can exhibit a partial or total alleviation of symptoms (for example, tumor load), or symoptoms can remain static following treatment according to the invention. The term “treatment” is intended to encompass prophylaxis, therapy and cure.

As used here, the phrase “5′ or 3′ sticky ends” is meant to refer to the 3′ and/or 5′ protruding ends of DNA or RNA that will bond with complementary sequences of bases. In certain embodiments, the RNA motifs have 5′ or 3′ sticky ends. In certain embodiments, the 5′ or 3′ sticky ends are located in the middle of a helix. According to the invention, the 5′ and 3′ sticky ends can be engineered to be used for self-assembly of the nanorings into an RNA nanotube.

### RNA and Nanostructure Design

RNA has a number of advantages for nanostructure design. Nanoparticle structures provide a size range that is large enough to avoid the problem of expulsion from the cell, but are small enough to avoid the problems of cell delivery often encountered with larger particles. RNA is the only biopolymer that can carry genetic information and has catalytic properties. RNA can naturally fold into complex motifs, and RNA motifs are capable of self-assembly. RNA has a natural functionality, for instance RNA can function as ribozymes or riboswitches. Further, RNA is advantageous in eliciting a very low immune response. Moreover, the construction of RNA into ordered, patterned superstuctures has a number of desirable characteristics, including the ability to self-assemble in precisely defined ways, the ability to undergo editing and replication, the ability to undergo controlled disassembly. RNA has versatility in function and structure. Functionally, RNA is the only biopolymer that can carry genetic information and that possesses catalytic properties. Structurally, RNA has predictable intra and intermolecular interactions with well-known structural geometry. The RNA strands that consist of adenine (A), guanine (G), cytosine (C), and uridine (U) can naturally, or can be programmed, to self-assemble via complementary base pairing. The helical region of RNA has a well-known nanometer scale structural geometry of 2.86 nm per helical turn with II base pairs and a 2.3 nm diameter. The self-assembly of RNA into complex structures can be facilitated via complementary base pairing or inter- and intra-molecular interactions of the different single stranded regions in the RNA, including internal bulges and loop motifs, and single-stranded overhangs or “sticky-ends”.

### RNA Synthesis

RNA molecules used to make the nanoparticles of the invention can be produced recombinantly or synthetically by methods that are routine for one of skill in the art. For example, synthetic RNA molecules can be made as described in US Patent Application Publication No.: 20020161219, or U.S. Pat. Nos. 6,469,158, 5,466,586, 5,281,781, or 6,787,305.

### RNA Self-Assembly

Small RNA structural motifs can code the precise topology of large molecular architectures. It has been shown that RNA structural motifs participate in a predictable manner to stabilize, position and pack RNA helices without the need of proteins (Chworos A et al., Science 306:2068-2072.2004). RNAI and RNAII are loop structures that interact in what is called a ‘kiss’ or ‘kissing’ complex (Lee et al., Structure 6:993-1005.1998). This contact facilitates the pairing of the RNAI and RNAII loops, until the two RNAs form a duplex. As such, the “kissing” interaction between RNAI and RNAII is one means of self-assembly between the RNA building blocks. The interaction between the RNAIi/RNAIIi complex involves all the bases in the base pairing, and dissociates nearly 7000 times more slowly than the wild-type complex.

The self-assembly of nanoparticles from RNA involves cooperative interaction of individual RNA molecules that spontaneously assemble in a predefined manner to form a larger two- or three-dimensional structure. Within the realm of self-assembly two main categories have been described: template and non-template (Lee et al. J Nanosci Nanotechnol. 2005 December; 5(12):1964-82). Template assembly involves interaction of RNA molecules under the influence of specific external sequence, forces, or spatial constraints such as RNA transcription, hybridization, replication, annealing, molding, or replicas. In contrast, non-template assembly involves formation of a larger structure by individual components without the influence of external forces. Examples of non-template assembly are ligation, chemical conjugation, covalent linkage, and loop/loop interaction of RNA, especially the formation of RNA multimeric complexes (Lee et al. 2005, as above).

In certain embodiments the RNA building blocks of the invention can self-assemble in buffer conditions suitable for RNA, and that can be determined by one of skill in the art.

In other certain embodiments, the nanostructures of the invention can be formed in a cell. In certain examples, the RNA sequence will be expressed in the cell and nano-ring formation will be observed via electron microscope tomography (EMT). To satisfy the EMT resolution requirements the minimal size of the nano-ring will be between 15 nm, 20 nm, 25 nm, 30, nm, 35 nm, 40 nm, 45 nm or more. In preferred embodiments, the minimal size of the nano-ring will be 25 nm. Moreover, in preferred embodiments, the nano-ring can further assemble into bundles, such as nanotubes, sheets, or clusters. In certain preferred embodiments, for overexpression in the cell the RNA nano-ring building blocks of one type, with RNAIi loop at one end and RNAIIi loop at another end, are used. The sequence and structure for an exemplary RNA nano-ring building block, with a projected particle size of 26.3 nm, and the ability to “bundle” via the sticky tem is shown below:

GcgcUAUCCAGgggaacggaugguucguucccUUGGGUAgcgcCCCG
UUCUCAAccgcaccgaaccauccggugcggUUGGGAACGGGaaCUC
UCUCUCUAGAGAGAGAG

Thus, based on these requirements, a sequence that can be used for Nano-ring overexpression in E. coli and folding directly in the cell has been designed. The following sequence is then incorporated into the E. coli genome and transcribed by T7 polymerase:

In certain preferred embodiments, the terminator sequence may not be used since the VSS1 Ribozyme should cleave the nano-sequence from the rest of the E. coli sequence.

### RNA Nanoparticles

RNA has been demonstrated to be an efficient nanoparticle. A bacteriophage phi29-encoded RNA (pRNA) has been reengineered to form dimmers, trimers, rods, hexamers, and 3D arrays several microns in size through interactions of interlocking loops (Shu, D.; Moll, W.-D.; Deng, Z.; Mao, C.; Guo, P. Nano Letters 2004, 4, (9), 1717-1723; Guo, P. J Nanosci Nanotechnol 2005, 5, (12), 1964-82). A nanoparticle, containing a pRNA trimer as a delivery vehicle was used to deliver siRNAs and receptor-binding aptamers, and has been demonstrated to block cancer development both in vitro in cell culture, and in vivo in mice (Khaled, A.; Guo, S.; Li, F.; Guo, P. Nano Lett 2005, 5, (9), 1797-808; Guo, S.; Huang, F.; Guo, P. Gene Ther 2006, 13, (10), 814-20). An H-shaped RNA molecular unit built from a portion of group I intron domain has been shown to form oriented filaments (Hansma, H. G.; Oroudjev, E.; Baudrey, S.; Jaeger, L. J Microsc 2003, 212, (Pt 3), 273-9; Nasalean, L.; Baudrey, S.; Leontis, N. B.; Jaeger, L. Nucleic Acids Res 2006, 34, (5), 1381-92). Further, specific RNA nano-arrangements based on HIV dimerization initiation site stem-loops were shown to be capable of thermal isomerization to alternative structures (Horiya, S.; Li, X.; Kawai, G.; Saito, R.; Katoh, A.; Kobayashi, K.; Harada, K. Nucleic Acids Res Suppl 2002, (2), 41-2; Horiya, S.; Li, X.; Kawai, G.; Saito, R.; Katoh, A.; Kobayashi, K.; Harada, K. Chem Biol 2003, 10, (7), 645-54.; Li, X.; Horiya, S.; Harada, K. J Am Chem Soc 2006, 128, (12), 4035-40). Small structural fragments found in the ribosome and HIV have been used in the design of artificial RNA building blocks, called tectoRNAs (Chworos, A.; Severcan, I.; Koyfinan, A. Y.; Weinkam, P.; Oroudjev, E.; Hansma, H. G.; Jaeger, L. Science 2004, 306, (5704), 2068-72). Each tectoRNA contains a right angle motif that forms a 90-degree angle between adjacent helices, two interacting hairpin loops at the end of each stem, and a 3′ “sticky stem”. The hairpin loops direct the formation of the tetramer via formation of specific noncovalent loop-loop interactions, called “kissing loops”, and the “sticky stems” further assemble tetramers into complex nanoarrays. In bionanotechnology, RNA-RNA interactions can guide precise deposition of gold nanoparticles (Bates, A. D.; Callen, B. P.; Cooper, J. M.; Cosstick, R.; Geary, C.; Glidle, A.; Jaeger, L.; Pearson, J. L.; Proupin-Perez, M.; Xu, C.; Cumming, D. R. Nano Lett 2006, 6, (3), 445-8). For example, self-assembling tectoRNA-ladders have been shown to induce a precise linear arrangement of cationic gold nanoparticles, demonstrating that RNA can control regular spacing of gold nanoparticles and can act as a nanocrown scaffold (Koyfinan, A. Y.; Braun, G.; Magonov, S.; Chworos, A.; Reich, N. O.; Jaeger, L. J Am Chem Soc 2005, 127, (34), 11886-7).

Design

The general approach used to create RNA nano-particles and nano-materials is to take known RNA structures, cut them into the building blocks, and reengineer single-stranded loops and regions to facilitate the desired self-assembly. The self-assembly of all the above discussed RNA building blocks into nanostructures is mediated by the complementarity of hairpin loops and loop receptors that form non-covalent RNA-RNA interactions. For precise assembly of the RNA building blocks, each of the corresponding complementary loop-loop interactions are uniquely reengineered.

Two main experimental approaches are used for programmable self-assembly of nucleic acids nanostructures (Jaeger, L.; Chworos, A. Curr Opin Struct Biol 2006, 16, (4), 531-43). The first is a single-step assembly, which is commonly used for DNA nanostructures (Chelyapov, N.; Brun, Y.; Gopalkrishnan, M.; Reishus, D.; Shaw, B.; Adleman, L. J Am Chem Soc 2004, 126, (43), 13924-5; Mathieu, F.; Liao, S.; Kopatsch, J.; Wang, T.; Mao, C.; Seeman, N. C. Nano Lett 2005, 5, (4), 661-5.). The second is a stepwise assembly, which has been commonly described for RNA nanostructures (Chworos, A.; Severcan, I.; Koyfman, A. Y.; Weinkam, P.; Oroudjev, E.; Hansma, H. G.; Jaeger, L. Science 2004, 306, (5704), 2068-72). In the single-step assembly approach, all molecules are mixed together followed by the slow cool annealing procedure. This is only possible if the target building block structure is the one that has the highest number of Watson-Crick base pairs and is therefore the most stable. This approach is, thus, based on the preferential folding of the building blocks at higher temperatures followed by the self-assembly of these building blocks through weaker interactions into final nanostructures at lower temperatures. However, usually there are many other possible structures that are only slightly less stable. In this case, the stepwise approach can be used where the building blocks are separately formed in the first step are then mixed together in the presence of high magnesium (Mg++) concentration to form a final nanostructure. This approach is more time consuming and the melting temperatures of the building blocks and the final nanostructure should be well separated:

The instant invention describes polyvalent RNA nanoparticles that comprise RNA motifs as building blocks. In certain embodiments, the building blocks comprise a motif that allows for non-covalent assembly between 2 or more building blocks.

A number of RNA motifs are available as building blocks, including but not limited to RNA I and/or RNA II motifs, kissing loops, RNA I inverse (RNA Ii) and/or RNA II inverse (RNA IIi) motifs. Numerous high-resolution RNA structures determined by NMR or X-ray crystallography can be separated into building blocks for design of new RNA nanoparticles and nanomaterials.

The polyvalent RNA nanoparticle of according to the invention can be in the shape of a ring, in the shape of a square or in the shape of a triangle; however it is to be understood that other geometries are possible. Accordingly, the ring, square, triangle or other shape comprises 1, 2, 3, 4, 5, 6, 7, 8, 9, 10, or more building blocks as described by the invention. In certain preferred embodiments of the invention, the ring comprises 6 building blocks that form a hexameric ring. In the hexameric ring, there is a 120-degree angle at the loop-loop interactions at the corners of the hexameric ring.

The RNA building blocks can contain hairpin loops. In certain specific example, each building block contains two or more hairpin loops. The hairpin loops are connected by a helix. The RNA building blocks can be held together by non-covalent loop-loop contacts. Further, the building blocks have 5′ or 3′ sticky ends, and in certain preferred embodiments of the invention, the 5′ or 3′ sticky ends are located in the middle of a helix.

### RNA Nanotubes

As described, the polyvalent RNA nanoparticles consist of building blocks, which have 5′ or 3′ sticky ends. These 5′ or 3′ sticky ends can be engineered as sticky ends for self-assembly of nanorings into an RNA nanotube. Advantageously, in certain embodiments, the RNA nanoparticles can be connected via complementary ends. This polyvalent RNA nanoparticle is capable of self-assembly into a nanotube. As discussed herein, in certain preferred embodiments, self-assembly may occur as a single-step process.

Conjugation to Nanoparticles

The polyvalent RNA nanoparticles can be used to deliver therapeutics, as diagnostic tools, or as delivery agents. Advantageously, the 5′ and 3′ sticky ends are positions for conjugation of one or more therapeutic, diagnostic, or delivery agents.

Exemplary potential applications of multi-functional nanoparticles of the invention in which 2, 3, 4, or more agents are coupled to a nanoparticle include using one or more agents to target a macromolecular structure or a cell and using the second one to alter the function/properties of the macromolecule or cell, e.g., using a protein to target a cell and using a toxin or cell death protein to kill the targeted cell, using an siRNA to silence genes, or using a fluorescent particle for visualization, or using a chemical or protein to target a protein within a complex and another one to alter the function of a different component of the complex.

Further exemplary potential applications of the multi-functional nanoparticles of the invention include use of the nanoparticles as riboswitch aptamers, ribozymes, or beacons.

Riboswitches are a type of control element that use untranslated sequence in an mRNA to form a binding pocket for a metabolite that regulates expression of that gene. Riboswitches are dual function molecules that undergo conformational changes and that communicate metabolite binding typically as either increased transcription termination or reduced translation efficiency via an expression platform.

Ribozymes catalyze fundamental biological processes, such as RNA cleavage by transesterification. The polyvalent RNA nanoparticles of the invention can be incorporated in to ribozymes using methods described in, for example, U.S. Pat. No. 6,916,653, incorporated by reference in its entirety herein.

A number of “molecular beacons” (often fluorescence compounds) can be attached to RNA nanoparticles of the invention to provide a means for signaling the presence of, and quantifying, a target analyte. Molecular beacons, for example, employ fluorescence resonance energy transfer-based methods to provide fluorescence signals in the presence of a particular analyte/biomarker of interest. In preferred embodiments, the term “molecular beacon” refers to a molecule or group of molecules (i.e., a nucleic acid molecule hybridized to an energy transfer complex or chromophore(s)) that can become detectable and can be attached to a nanoparticle under preselected conditions. Similarly, amplifying fluorescent polymers (AFPs) can be utilized in the present invention. An AFP is a polymer containing several chromophores that are linked together. As opposed to isolated chromophores that require 1:1 interaction with an analyte in conventional fluorescence detection, the fluorescence of many chromophores in an AFP can be influenced by a single molecule. For example, a single binding event to an AFP can quench the fluorescence of many polymer repeat units, resulting in an amplification of the quenching. Quenching is a process which decreases the intensity of the fluorescence emission. Molecular beacons and AFPs, including their methods for preparation, that can be used in the present invention are described in numerous patents and publications, including U.S. Pat. No. 6,261,783.

Any protein can be coupled to nanoparticles. For instance, glycoproteins are most easily coupled, as they can be oxidized to generate an active aldehyde group. Other proteins can be coupled via their —COOH group(s) but with lower efficiency. However, other means known in the art, such as di-imide reagents, e.g. carbodiimide can be used to couple proteins lacking sugars to the nanoparticles.

Polyethylene Glyocol (PEG) chains can be conjugated to the nanoparticles. PEG chains render the nanotubes highly water-soluble. PEG-phospholipids (PEG-PL) have been used in the formation of micelles and liposomes for drug delivery (Adlakha-Hutcheon, G.; Bally, M. B.; Shew, C. R.; Madden, T. D. Nature Biotech. 1999, 17, 775-779; Meyer, O.; Kirpotin, D.; Hong, K.; Sternberg, B.; Park, J. W.; Woodle, M. C.; Papahadjopoulos, D. J. Biol. Chem. 1998, 273, 15621-15627; Papahadjopoulos, D.; Allen, T. M.; Gabizon, A.; Mayhew, E.; Matthay, K.; Huang, S. K.; Lee, K. D.; Woodle, M. C.; Lasic, D. D.; Redemann, C.; Martin, F. J. Proc. Nat. Acad. Sci. USA. 1991, 88, 11460-11464).

Functional groups can be coupled to the nanoparticle, for instance the functional group can be a reactive functional group. Suitable functional groups include, but are not limited to, a haloacetyl group, an amine, a thiol, a phosphate, a carboxylate, a hydrazine, a hydrazide an aldehyde or a combination thereof. Other functional groups include groups such as a reactive functionality or a complementary group. In addition, RNA functional groups can be attached, as for example ribozymes or riboswitch aptamers.

The nanoparticle can be used for attachment of small molecules for specific interactions with nucleic acids, carbohydrates, lipids, proteins, antibodies, or other ligands.

The nanoparticle can have dyes attached. The dye is can be a fluorescent dye, or a plurality of fluorescent dyes. Suitable dyes include, but are not limited to, YOYO-1, JOJO-1, LOLO-1, YOYO-3, TOTO, BOBO-3, SYBR, SYTO, SYTOX, PicoGreen, OliGreen, and combinations thereof. Other dyes include, thiazole orange, oxazole yellow, or non-intercalating dyes such as fluorescein, rhodamine, cyanine or coumarin based dyes, and combinations thereof. Other suitable dyes include, but are not limited to, 4-acetamido-4′-isothiocyanatostilbene-2,2′disulfonic acid; acridine and derivatives: acridine, acridine isothiocyanate; 5-(2′-aminoethyl)aminonap-hthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; BODIPY; Brilliant Yellow; coumarin and derivatives: coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,-2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amin-ofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid; terbium chelate derivatives; Cy 3; Cy 5; Cy 5.5; Cy 7; IRD 700; IRD 800; La Jolla Blue; phthalocyanine; and naphthalo cyanine. Suitable dyes for use in the nanoparticles of the present invention include, without limitation, a family of homodimeric cyanine DNA intercalating dyes from Molecular Probes that cover the visible spectrum, such as YOYO-1 (488/509), JOJO-1 (532/545), LOLO-1 (565/579), and YOYO-3 (612/631), SYBR-101 (488/505) and SYTO-62 (652/676). Given sufficient detection SN, dyes are mixed in various ratios in a single particle such that, for example, different fluorescence spectra are obtained from mixtures of just 2 dyes.

According to the invention, one or more therapeutic, diagnostic, or delivery agents are directly included in the building block sequences. In certain embodiments, the delivery agent can be a targeting agent. Targeting agents are used to direct the nanoparticle to a tissue or cell target. An exemplary embodiment of a targeting agent is an antibody. For example, antibodies suitable for use as targeting agents in the present invention include antibodies directed to cell surface antigens which cause the antibody-nanoparticle complex to be internalized, either directly or indirectly. For example, in the treatment of cancer, suitable antibodies include antibodies to CD33 and CD22. CD33 and CD22 that are over-expressed and dimerized on lymphomas.

A therapeutic agent can be coupled to the nanoparticle for prevention or treatment of a disease or condition. The therapeutic agent is selected from, but not limited to: siRNA, aptamers, oligonucleotides, antisense RNA, chemotherapeutic agents, ribozymes, and, fluorescent beads, heavy metals, radioisotopes, quantum dots, and molecular beacons.

A delivery agent can be coupled to the nanoparticle. The delivery agent is selected from, but not limited to: liposomes, antibodies, polyamines, polyethylene glycol. In addition, the delivery vehicle may be the naked RNA particle itself.

A wide variety of particles sizes are suitable for the present invention. In certain aspects, the particle has a diameter of about 10 nanometers to about 10 microns. Preferably the particle diameter is about 10 to 700 nanometers, and more preferably, the diameter of about 10 nanometers to about 100 nanometers.

The polyvalent RNA nanoparticle or the polyvalent RNA nanotube as described herein has a number of uses. For example, the polyvalent RNA nanoparticle or the polyvalent RNA nanotube can be used in drug delivery, imaging, nanocircuits, cell growth surfaces, medical implants, medical testing, or gene therapy.

In one particular embodiment, the polyvalent RNA nanoparticle or the polyvalent RNA nanotube as described can be used in biological meshes. In one exemplary embodiment, the invention as described herein may find use as a biosensor in, for example, pathogen detection. In one particular embodiment, self-assembling nano-meshes are used to attach biosensors for pathogen detection or for x-ray crystallography by placing multiple copies of a protein or functional RNAs, for example, on the mesh. Biosensors for pathogen detection are advantageously employed in bioterrorism capacities.

In another exemplary embodiment, nanotubes of the invention, as described herein, are employed as skeletons or scaffolds for tissue growth.

These uses are exemplary, and not considered to be limiting.

### Compositions

The invention, in part, pertains to a drug delivery composition comprising the polyvalent RNA nanoparticle as described herein. The drug delivery composition of the invention can gain entry into a cell or tissue.

Advantageously, the drug delivery composition of the invention provides for a more controlled delivery of an active agent, especially a therapeutic agent, to a site of action at an optimum rate and therapeutic dose. Thus, improvements in therapeutic index may be obtained by modulating the distribution of the active ingredient in the body. Association of the active ingredient with a delivery system enables, in particular, its specific delivery to the site of action or its controlled release after targeting the action site. By reducing the amount of active ingredient in the compartments in which its presence is not desired, it is possible to increase the efficacy of the active ingredient, to reduce its toxic side effects and even modify or restore its activity.

It is understood by one of skill in the art that changing the base composition of RNA changes the half-life of RNA and thus the release of RNA from the composition. For instance, the composition can be modified to consist of fast release, slow release or a staged release of polyvalent RNA nanoparticle.

In certain preferred embodiments, the drug delivery composition can comprise a second therapeutic agent. In some embodiments, the composition comprising nanoparticles and the second therapeutic agent are administered simultaneously, either in the same composition or in separate compositions. In some embodiments, the nanoparticle composition and the second therapeutic agent are administered sequentially, i.e., the nanoparticle composition is administered either prior to or after the administration of the second therapeutic agent. The term “sequential administration” as used herein means that the drug in the nanoparticle composition and the second agent are administered with a time separation of more than about 15 minutes, such as more than about any of 20, 30, 40, 50, 60 or more minutes. Either the nanoparticle composition or the chemotherapeutic agent may be administered first. The nanoparticle composition and the chemotherapeutic agent are contained in separate compositions, which may be contained in the same or different packages. In some embodiments, the administration of the nanoparticle composition and the second therapeutic agent are concurrent, i.e., the administration period of the nanoparticle composition and that of the second therapeutic agent overlap with each other. In some embodiments, the administration of the nanoparticle composition and the second therapeutic agent are non-concurrent. For example, in some embodiments, the administration of the nanoparticle composition is terminated before the second therapeutic agent is administered.

In some embodiments, the administration of the second therapeutic agent is terminated before the nanoparticle composition is administered. Administration may also be controlled by designing the RNA nanoparticle or nano-tube to have different half lives. Thus, particle dissolution would be controlled by a timed release based upon variations in designed RNA stability.

The second therapeutic agent is selected from, but not limited to chemotherapeutic agents, cardiovascular drugs, respiratory drugs, sympathomimetic drugs, cholinomimetic drugs, adrenergic or adrenergic neuron blocking drugs, analgesics/antipyretics, anesthetics, antiasthmatics, antibiotics, antidepressants, antidiabetics, antifungals, antihypertensives, anti-inflammatories, antianxiety agents, immunosuppressive agents, immunomodulatory agents, antimigraine agents, sedatives/hypnotics, antianginal agents, antipsychotics, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterials, antivirals, antimicrobials, anti-infectives, bronchodialators, hormones, hypoglycemic agents, hypolipidemic agents, proteins, peptides, nucleic acids, agents useful for erythropoiesis stimulation, antiulcer/antireflux agents, antinauseants/antiemetics and oil-soluble vitamins, or combinations thereof.

When the second therapeutic agent is a chemotherapeutic agent, the chemotherapeutic agent is selected from, but not limited to, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfomithine, elliptinium acetate, etoglucid, flutamide, hydroxyurea, lentinan, phenarnet, podophyllinic acid, 2-ethylhydrazide,.razoxane, spirogermanium, tamoxifen, taxotere, tenuazonic acid, triaziquone, 2,′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinyispermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor I-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

Reference to a chemotherapeutic agent herein applies to the chemotherapeutic agent or its derivatives and accordingly the invention contemplates and includes either of these embodiments (agent; agent or derivative(s)). “Derivatives” or “analogs” of a chemotherapeutic agent or other chemical moiety include, but are not limited to, compounds that are structurally similar to the chemotherapeutic agent or moiety or are in the same general chemical class as the chemotherapeutic agent or moiety. In some embodiments, the derivative or analog of the chemotherapeutic agent or moiety retains similar chemical and/or physical property (including, for example, functionality) of the chemotherapeutic agent or moiety.

The invention also relates to pharmaceutical or diagnostic compositions comprising the nanoparticles of the invention and a pharmaceutically acceptable carrier. The phrase “pharmaceutically acceptable carrier” is art recognized and includes a pharmaceutically acceptable material, composition or vehicle, suitable for administering compounds used in the methods described herein to subjects, e.g., mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in Remington's Pharmaceutical Sciences, Mack Publishing Company, a standard reference text in this field.

### Methods of Treatment

The methods of the invention encompass method of treating or preventing diseases or disorders by administering to subjects in need thereof an effective amount of a polyvalent RNA nanoparticle or nanotube as described herein. Accordingly, a number of diseases or disorders are suitable for treatment according to the methods of the invention. Examples include, but are not limited to, Adenoma, Ageing, AIDS, Alopecia, Alzbeimer's disease, Anemia, Arthritis, Asthma, Atherosclerosis, Cancer, Cardiac conditions or disease, Diabetes mellitus, Foodborne illness, Hemophilia A-E, Herpes, Huntington's disease, Hypertension, Headache, Influenza, Multiple Sclerosis, Myasthenia gravis, Neoplasm, Obesity, Osteoarthritis, Pancreatitis, Parkinson's disease, Pelvic inflammatory disease, Peritonitis, Periodontal disease, Rheumatoid arthritis, Sepsis, Sickle-cell disease, Teratoma, Ulcerative colitis, and Uveitis.

The methods of the invention further encompass diagnostics.

The methods may be practiced in an adjuvant setting. “Adjuvant setting” refers to a clinical setting in which, for example, an individual has had a history of a proliferative disease, particularly cancer, and generally (but not necessarily) been responsive to therapy, which includes, but is not limited to, surgery (such as surgical resection), radiotherapy, and chemotherapy. However, because of their history of the proliferative disease (such as cancer), these individuals are considered at risk of development of the disease. Treatment or administration in the “adjuvant setting” refers to a subsequent mode of treatment. The degree of risk (i.e., when an individual in the adjuvant setting is considered as “high risk” or “low risk”) depends upon several factors, most usually the extent of disease when first treated. The methods provided herein may also be practiced in a neoadjuvant setting, i.e., the method may be carried out before the primary/definitive therapy. Thus, in some embodiments, the individual has previously been treated. In other embodiments, the individual has not previously been treated. In some embodiments, the treatment is a first line therapy.

### Methods of Delivery

The nanoparticle compositions described herein can be administered to an individual (such as human) via various routes, such as parenterally, including intravenous, intra-arterial, intraperitoneal, intrapulmonary, oral, inhalation, intravesicular, intramuscular, intra-tracheal, subcutaneous, intraocular, intrathecal, or transdermal. For example, the nanoparticle composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In some embodiments, the nanoparticle composition is administrated intravenously. In some embodiments, the nanoparticle composition is administered orally.

The dosing frequency of the administration of the nanoparticle composition depends on the nature of the therapy and the particular disease being treated. For example, dosing frequency may include, but is not limited to, once daily, twice daily, weekly without break; weekly, three out of four weeks; once every three weeks; once every two weeks; weekly, two out of three weeks.

The administration of nanoparticles may be carried out at a single dose or at a dose repeated once or several times after a certain time interval. The appropriate dosage varies according to various parameters, for example the individual treated or the mode of administration.

The dosing frequency of the nanoparticle composition or the nanoparticle composition and the second therapeutic agent may be adjusted over the course of the treatment, based on the judgment of the administering physician.

When administered separately, the nanoparticle composition and the second therapeutic agent can be administered at different dosing frequency or intervals. For example, the nanoparticle composition can be administered weekly, while a second agent can be administered more or less frequently. In some embodiments, sustained continuous release formulation of the nanoparticle and/or second agent may be used. Various formulations and devices for achieving sustained release are known in the art. The doses required for the nanoparticle composition and/or the second agent may (but not necessarily) be lower than what is normally required when each agent is administered alone. Thus, in some embodiments, a subtherapeutic amount of the drug in the nanoparticle composition and/or the second agent are administered. “Subtherapeutic amount” or “subtherapeutic level” refer to an amount that is less than the therapeutic amount, that is, less than the amount normally used when the drug in the nanoparticle composition and/or the second agent are administered alone. The reduction may be reflected in terms of the amount administered at a given administration and/or the amount administered over a given period of time (reduced frequency).

A combination of the administration configurations described herein can be used. The combination therapy methods described herein may be performed alone or in conjunction with another therapy, such as surgery, radiation, chemotherapy, immunotherapy, gene therapy, and the like. Additionally, a person having a greater risk of developing the disease to be treated may receive treatments to inhibit or and/or delay the development of the disease.

The dose of nanoparticle composition will vary with the nature of the therapy and the particular disease being treated. The dose should be sufficient to effect a desirable response, such as a therapeutic or prophylactic response against a particular disease.

Appropriate doses will be established by persons skilled in the art of pharmaceutical dosing such as physicians.

### Examples

It should be appreciated that the invention should not be construed to be limited to the examples that are now described; rather, the invention should be construed to include any and all applications provided herein and all equivalent variations within the skill of the ordinary artisan.

### Example 1

### Nanostructure Design—the Loop-Loop Structure

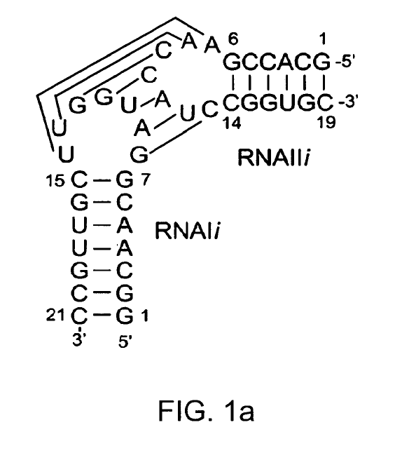

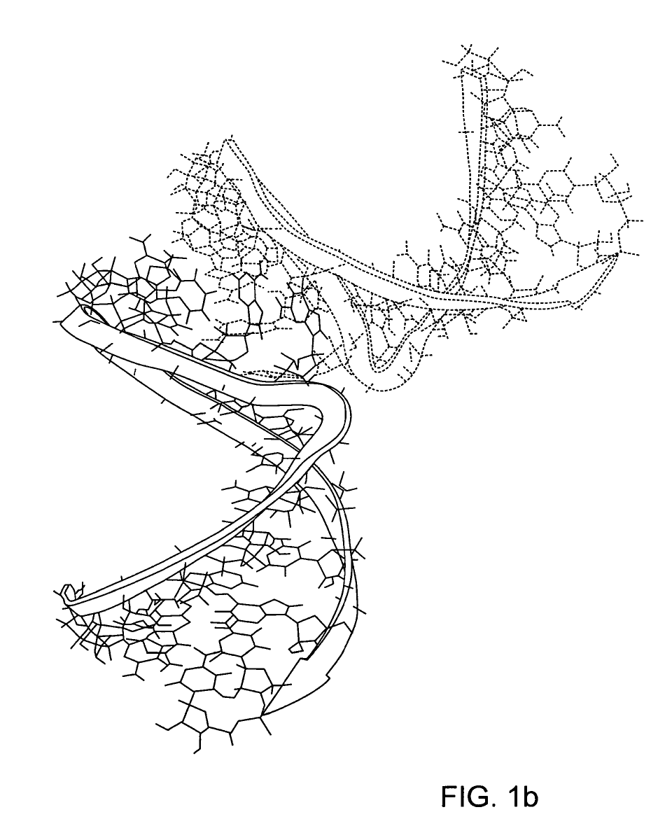

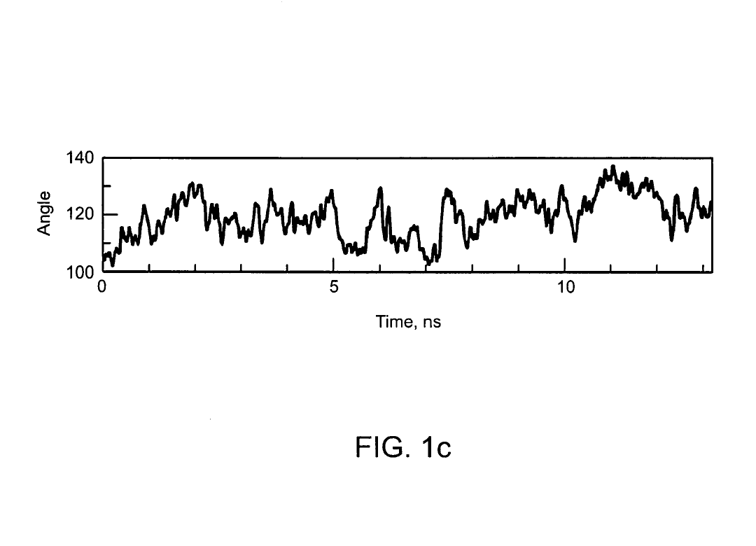

Several important points must be considered in nanostructure design. First, the building blocks' formation has to be more energetically favorable than non-covalent loop-loop interactions. Second, the intermolecular associations that facilitate the self-assembly of building blocks into a nanostructure have to be Watson-Crick complementary. Third, the nanostructure is advantageously designed using as few types of building blocks as possible to reduce the experimental and fabrication costs. Numerous high-resolution RNA structures determined by NMR or X-ray crystallography can be separated into building blocks for design of new RNA nanoparticles and nanomaterials. Since loop-loop interactions are commonly used for self-assembly of separate building blocks into superstructures, all known loop-loop complexes were examined. The NMR-determined loop-loop structure of RNAI/RNAII inverse (RNA Ii/RNA IIi), as shown in FIG. 1(a-c), has a distinct bend at the loop-loop helix which can represent corners of the hexameric ring. RNA I and RNA II are antisense and sense plasmid-encoded transcripts which control the replication of the ColE1 plasmid of Escherichia coli via duplex formation. This structure has two important structural features: it exhibits a unique bend, and has phosphate clusters flanking the major groove of the loop-loop helix. These non-sequence specific structural features can be recognized by the “RNA one modulator” (ROM) protein. Replication of the ColE1 plasmid of Escherichia coli is regulated by the interaction of sense and antisense plasmid-encoded transcripts. The antisense RNA I negatively regulates the replication of the plasmid by duplex formation with complementary RNA II. The interaction is initiated by the formation of a double helix between seven-nucleotide loops from each RNA and is stabilized by binding of the ROM protein. The ROM protein is thought to recognize a specific RNA structure, regardless of sequence (Lee et al. Structure. Aug 15, 1998;6(8):993-1005). The binding of the ROM protein to the kissing loop structure reduces the equilibrium dissociation constant of the complex (Tomizawa, J. J Mol Biol 1990, 212, (4), 695-708). The inversion of the RNA I/RNA II loop sequences relative to the wild-type sequences makes the complex dissociate 7000 times more slowly than the wild-type, and increases the quality of NMR spectra (Eguchi, Y.; Tomizawa, J. J Mol Biol 1991, 220, (4), 831-42). The RNA I/RNA II inverse complex was subjected to extensive explicit solvent molecular dynamics simulations to check the flexibility and relative stability. During the course of the simulations all seven base pairs between the loops of the RNA Ii and RNA IIi were maintained. The measurement of the angle between the helical stems, as shown in FIG. 1c shows that the structure is stable and fluctuates around a 120 degree kink at the loop-loop interactions. Thus, this loop-loop structure is very well fit to represent the corners of the hexameric ring.

### Example 2

### Nanostructure Design—the Building Blocks

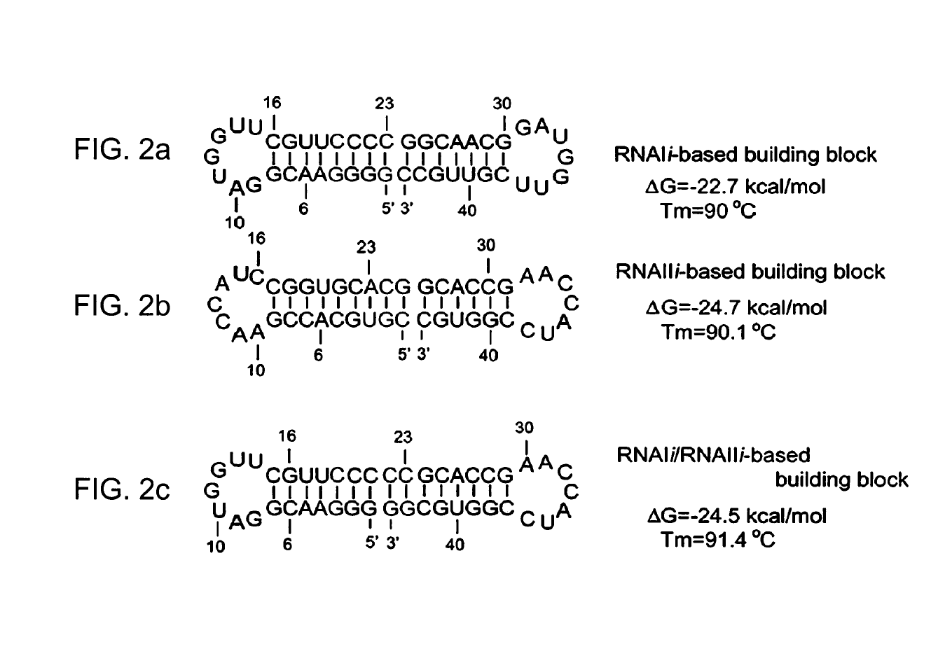

The next step is to create the building blocks using these RNA Ii and RNA IIi loops. The simplest approach is to use a helix to connect the desired loops. RNA sequences were designed that form helices capped with the two loops that are based on the RNA Ii and RNA IIi sequences (FIG. 2). There are two possibilities for forming a ring using RNA Ii and RNA IIi loop-loop interactions. The first possibility is to use two types of building blocks, one building block has the RNA Ii sequence in both loops, as shown in FIG. 2a, and the other building block has the RNA IIi sequence in both loops, as shown in FIG. 2b. The second possibility is to use a single type of building block, where one loop has the RNA Ii sequence and the other loop has the RNA IIi sequence, as shown in FIG. 2c. Using the secondary structure prediction method, MFOLD, modified several base pairs were modified. The modified base pairs are highlighted with arrows in FIG. 2. The base pairs were modified in each building block in order to obtain the single most energetically favorable structural conformation for a given sequence and thus preclude other conformational folds.

### Example 3

### Nanostructure Design—Modeling the RNA Nanoring

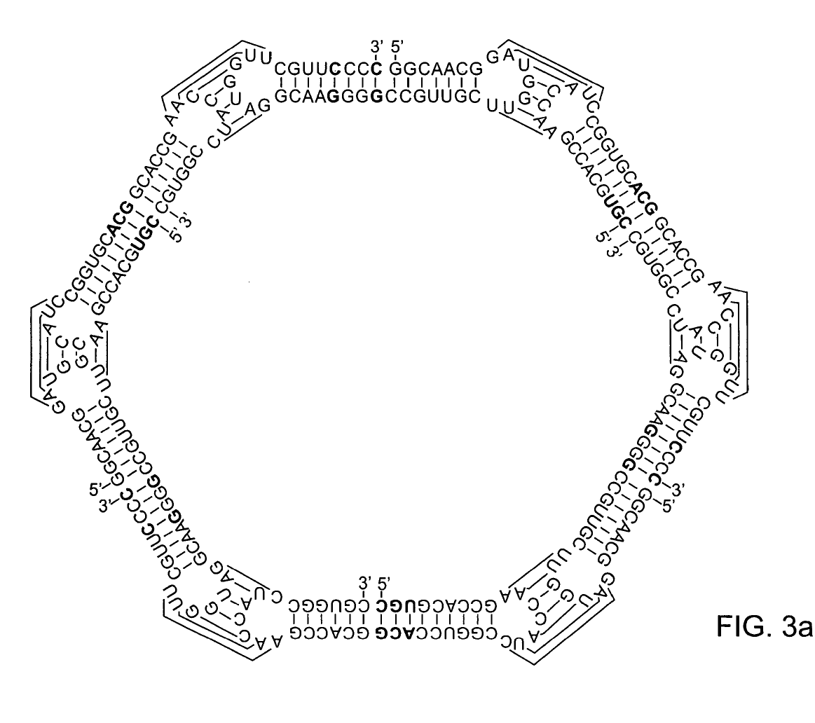

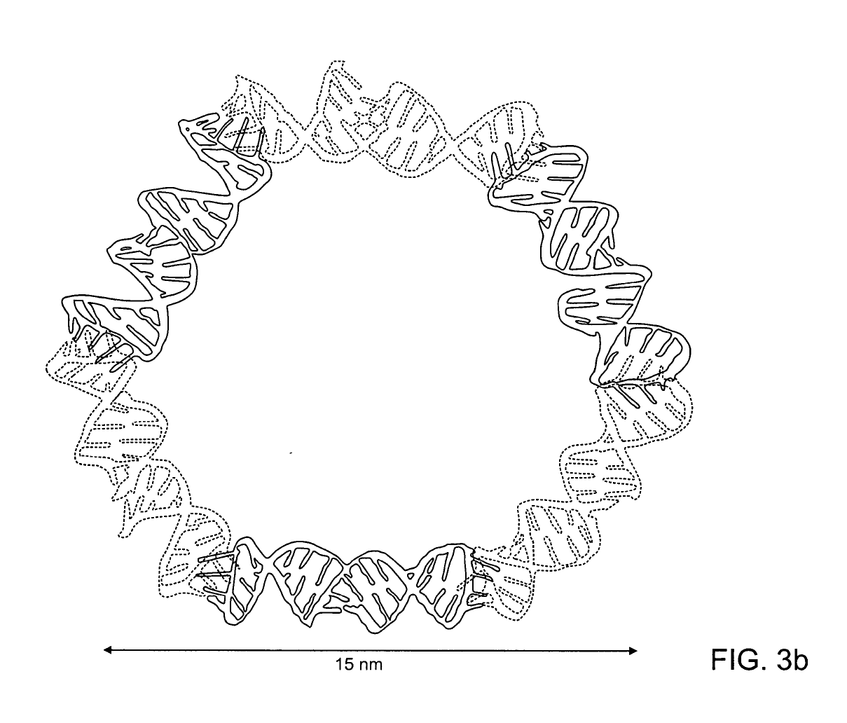

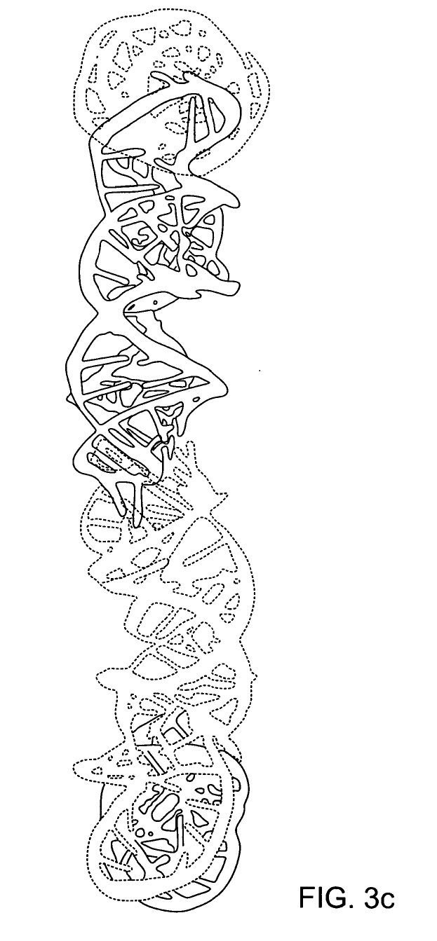

To model the RNA nanoring the original coordinates of the RNA I/RNA II inverse complex were imported six times into the INSIGHT II program. A helical template was used to assemble these six corner complexes into a nanoring. This helical template has common base pairs with the end of RNA Ii stem at one end and the end of RNA IIi stem at another end. The template is used to align the corner complexes by superimposing the corresponding common base pairs in the template and RNA Ii stem or RNA IIi stem. To produce a complete helical turn three extra base pairs and 3′ and 5′ ends were introduced in the middle of the helical region of a template. The template nucleotides were then removed leaving a meaningful structure. The rest of the building blocks were fitted in a similar way until a complete nanoring was built. The resultant structure was subjected to slow constrained equilibration and 8 ns implicit molecular dynamics simulations and was shown to possess no violations and was able to maintain stability and planarity. The secondary and tertiary structure of the designed nanoring is shown in FIG. 3. FIG. 3 shows the minimum size of the nanoring measures 15 nanometers (nm) diagonally. The size of the ring can be easily expanded by adding full helical turns to the stems of the building blocks. In this case, the size of the nano-ring can be easily predicted using following formula:

Z≈15+2(N*2.8)

where Z is the size of the nano-ring in nm and N is the number of additional helical turns in the initial building block.

### Example 4

### Incorporating siRNA into RNA Building Blocks

There are a number of potential possibilities for the use of the nano-ring as described herein for delivery of therapeutic RNAs. One, for example, is to directly include RNA therapeutics into the building block sequences since the nanoparticle is all-RNA. Another is to fuse therapeutics onto the 3′ and 5′ ends of the nanoparticles building blocks.

The structure of a siRNA consists of a short 20-25-nucleotide double-strand of RNA with two nucleotide 3′ overhangs at both ends. These siRNA duplexes can be generated in situ by cleavage of artificial short hairpin RNA (shRNA). This shRNA produces a single transcript, which can be processed into a functional siRNA via an RNA polymerase III promoter (Dykxhoorn, D. M.; Lieberman, J. Annu Rev Med 2005, 56, 401-23). Thus, the building blocks as described herein can possess siRNA sequences in the helices closed by RNA Ii or RNA IIi loops.

There are several factors that are important for efficient design of shRNA, including a lack of four consecutive adenines (A) to avoid premature transcription termination, an AA flanking dimer on the 5′ end, a G or C residue after the AA dimer, and sense strand base preferences at positions 3(A), 10(U), 13(A), and 19 (A) (Izquierdo, M. Cancer Gene Ther 2005, 12, (3), 217-27). The above-discussed requirements can be taken into account during incorporation of a particular siRNA duplex into the building blocks via use of the RNA secondary structure prediction methods. Thermodynamic experiments indicate that only the sequences of the loops and the first two base pairs in both of the RNA Ii and RNA IIi stems are important for the RNA Ii/RNA IIi complex stability (Gregorian, R. S., Jr.; Crothers, D. M. J Mol Biol 1995, 248, (5), 968-84). However, the rest of the stem's nucleotides do not crucially influence the stability of the complex. Thus, the sequences of the helical part of the building blocks can be engineered into the desired siRNA sequences, capping them with RNA Ii or RNA IIi loops. The all-RNA nanoring with building blocks containing siRNAs and held together through non-covalent interactions can be used for efficient delivery of therapeutic RNAs. The formation of a double stranded hexamer with no single stranded regions makes the nanoparticle more resistant to RNAase digestion.

### Example 5

### Nanostructure Design—Polyvalent Nanoparticles

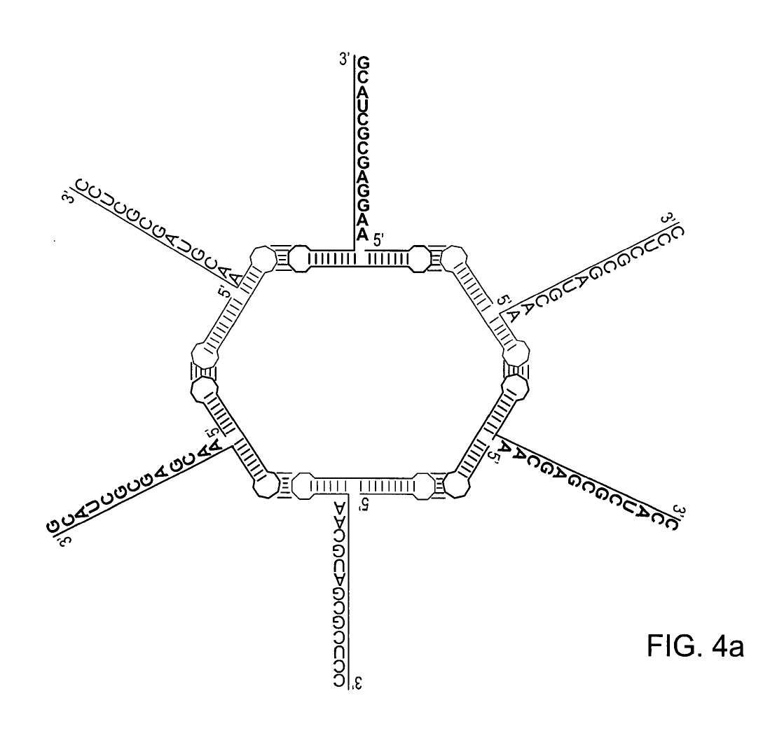

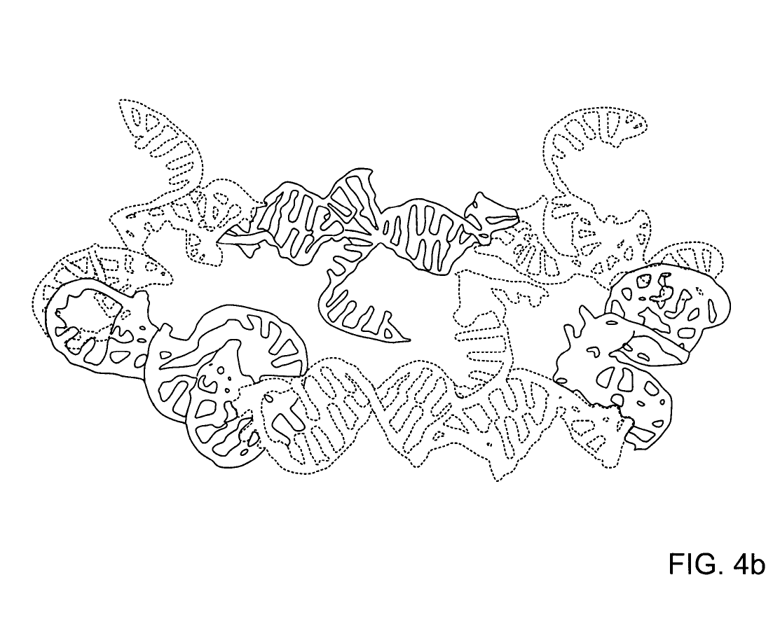

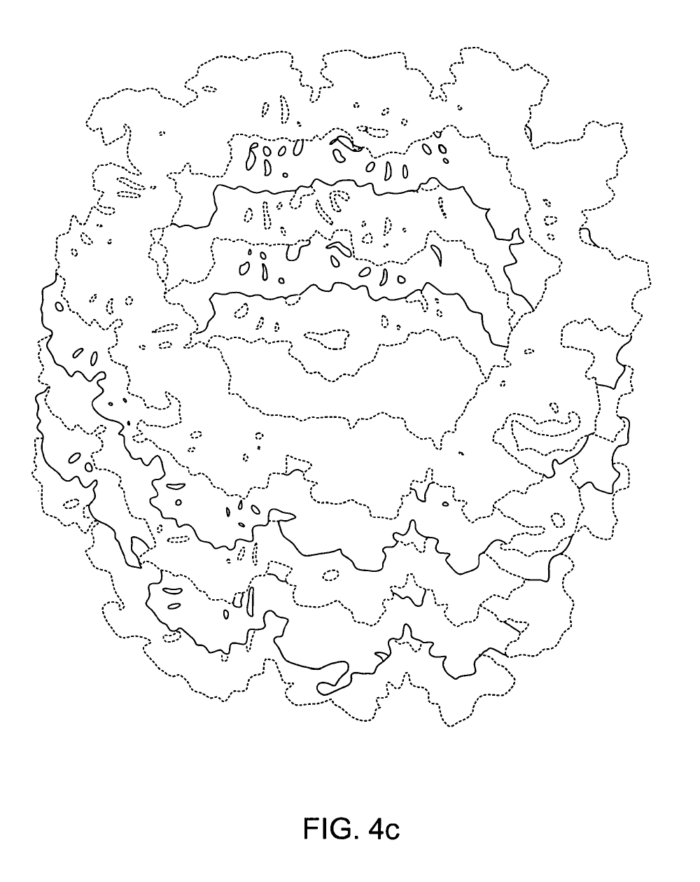

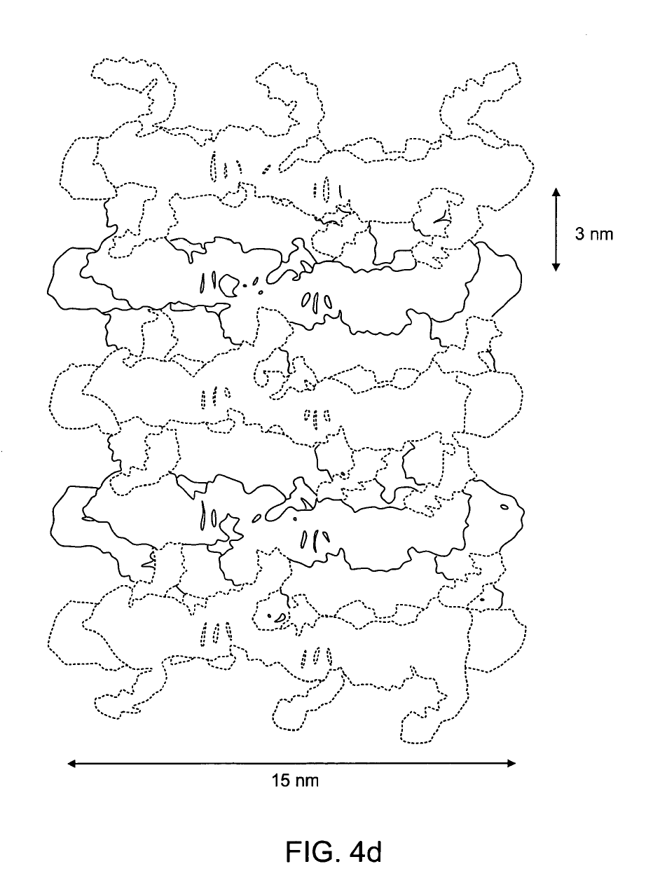

As described herein, the nanorings of the invention are suited for conjugation with agents, for example therapeutic, diagnostic, or delivery agents. As such, the constructed nanoring can be used as polyvalent nanoparticle with six available positions at the 3′ or 5′ ends to carry multiple components for cell recognition and therapy, including siRNAs, ribozymes, aptamers, cell surface ligands, heavy metals, quantum dots, and fluorescent beads. It has been shown that connecting the 3′ and 5′ ends of pRNA building blocks with a ribozyme or siRNA does not interfere with its folding process and function (Guo, P. J Nanosci Nanotechnol 2005, 5, (12), 1964-82). Thus, the 3′ and 5′ extensions of the nanoring building blocks can potentially be used to bind non-covalently or covalently-linked therapeutic RNA sequences, proteins, and molecular beacons. In this design, the 3′ tail is extended using two adenine nucleotides which have a tendency to turn away from the stem and 11 bases are added to create a full helical turn, as shown in FIG. 4a. Breaks (3′ and 5′ ends) in the building blocks' helices were made in specific places where the 3′ end of the RNA Ii-based building block is facing up and the 3′ end of RNA IIi-based building block is facing down (FIG. 4b). If desired, all the tails can face in the same direction by breaking the helices of both building blocks in the same place.

Moreover, these 3′ ends can be engineered to act as sticky, interacting tails that can be programmed to self-assemble into a superstructure. The 3′ tails of the RNA Ii-based building blocks and RNA IIi-based building blocks can be designed to be complementary to each other, thus making the sticky ends that face up from the nanoring to interlock with the sticky ends that face down from another nano-ring. Thus, these RNA rings are capable of self-assembly into a nanotube structure (FIG. 4c,d) with controllable dimensions.

Taken together, the examples describe a shape-based in silico approach to build and optimize an RNA nanoring. The RNA nanoring is built from simple helical building blocks of either one or two types. These building blocks can self-assemble through the non-covalent intermolecular interactions which are well-known for their stability and are based on RNA Ii/RNA IIi interactions. The size of nanoring can be easily extended by incorporation of as many helical turns as needed into the building block helical sequences. Moreover, the incorporation of “sticky ends” into the design allows the design of an RNA nanotube. Advanced simulation methods have been employed, including secondary structure optimization and explicit and implicit molecular dynamics simulations, to produce a possible design of a nanoparticle that is able to carry siRNAs.

### Example 6

### Nanostructure Formation in the Cell

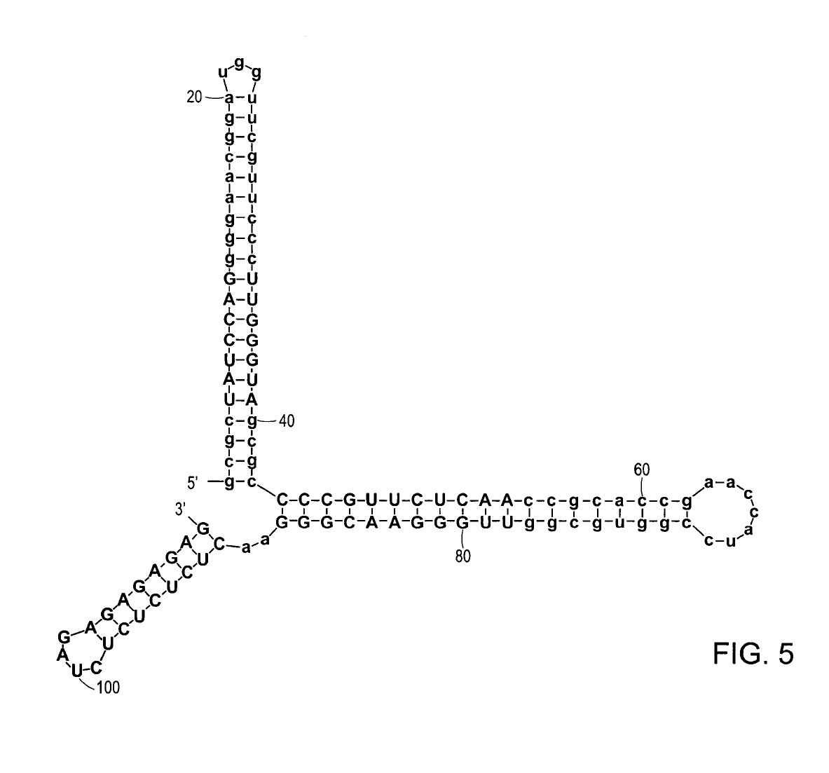

In another example, the nanostructures can be formed in a cell. The RNA sequence will be expressed in the cell and Nano-ring formation will be observed via electron microscope tomography (EMT). To satisfy the EMT resolution requirements the minimal size of the nano-ring will be between at least 25 nm, 30, nm, 35 nm, 40 nm, 45 nm or more. In certain preferred embodiments, the minimal size of the nano-ring will be 25 nm. Moreover, in preferred embodiments, the nanoring can further assemble into bundles, such as nanotubes, sheets, or clusters. In certain preferred embodiments, for overexpression in the cell the RNA nanoring building blocks of one type, with an RNAIi loop at one end and an RNAIIi loop at another end, are used. FIG. 5 shows an exemplary secondary structure of the RNA nano-ring building block used in this study. The structure was folded via the secondary structure prediction program MFOLD.

The general sequence for any type of the Nanoring building block expression in the cell is shown below. The bolded words represent variable sequences, which are dependent upon the desired nanoring building block sequence.

(T7PROMOTER) GAAATTAATACGACTCACTATAGGG
(Linker) AGACCACAACGGTTTCCCTCTAGAATAATTTTGTTTAACT
TTAAGAAGGAGATATACAT
(HAMMERHEAD RIBOZYME) GGGAGAGCAUAC
11 bases of complementary sequence to the 5′ end
of the Nano-ring building block sequence
CUGAUGAGUCCGUGAGGACGAAACGGUACCCGGUACCGUC
Nano-ring Sequence (SEQUENCE FOR VSS1 RIBOZYME)
AAGGGCGUCGUCGCCCCGAGGAUC
(sequence for terminator) CTAGCATAACCCCTTGGGGCCTCT
AAACGGGTCTTGAGGGGTTTTTT

The sequence and structure for an exemplary RNA nano-ring building block, with a projected particle size of 26.3 nm, and the ability to “bundle” via the sticky stem is shown below.

(SEQ ID NO: 3)

GcgcUAUCCAGgggaacggaugguucguucccUUGGGUAgcgcCCCG
UUCUCAAccgcaccgaaccauccggugcggUUGGGAACGGGaaCUC
UCUCUCUAGAGAGAGAG

Thus, in certain examples, the final design of the sequence used for overexpression of nanoring building blocks in the cell can be, but is not limited to, the following:

(SEQ ID NO: 4)

GAAATTAATACGACTCACTATAGGGAGACCACAA
CGGTTTCCCTCTAGAATAATTTTGTTTAACTTTAAGAAGG
AGATATACATGGGAGAGCAUACCUGGAUAGCGCCUGAU
GAGUCCGUGAGGACGAAACGGUACCCGGUACCGUCGCG
CUAUCCAGGGGAACGGAUGGUUCGUUCCCUUGGGUAGC
GCCCCGUUCUCAACCGCACCGAACCAUCCGGUGCGGUU
GGGAACGGGAACUCUCUCUCUAGAGAGAGAGAAGGGC
GUCGUCGCCCCGAGGAUCCTAGCATAACCCCTTGGGGCC
TCTAAACGGGTCTTGAGGGGTTTTTT

In certain embodiments, the RNAIi sequences comprises cggaugguucg (SEQ ID NO: 1) and the RNAIIi sequence comprises RNAIIi comprises cgaaccauccg (SEQ ID NO: 2).

### Materials and Methods of the Invention

The results reported herein were obtained using the following Materials and Methods:

Three-dimensional structure. The starting coordinates of RNA Ii/RNA IIi complex were taken from the PDB databank (PDB id 2bj2.pdb) (Lee, A. J.; Crothers, D. M. Structure 1998, 6, (8), 993-1005). The structures were extended and manipulated into a nano-ring and nanotube using Insight II and DSViewer INC.

RNA Secondary Structure Optimization. RNA secondary structure can be predicted from single sequence by free energy minimization methods. The method relies on empirical nearest neighbor free energy parameters, where the stability of each base pair or loop depends on the identity of nucleotides in the motif and in the adjacent base pairs. The RNA building blocks folding and thermodynamic parameters were determined by the program called MFOLD (Zuker, M.; Mathews, D. H.; Turner, D. H., Algorithms and Thermodynamics for RNA Secondary Structure Prediction: A Practical Guide in RNA Biochemistry and Biotechnology. Kluwer Academic Publishers: 1999). Alternatively, MPGAfold (Shapiro, B. A.; Bengali, D.; Kasprzak, W.; Wu, J. C. J Mol Biol 2001, 312, (1), 2744) can be used to determine folding pathways of more complex building blocks.

Molecular Dynamics Simulations. Molecular dynamics (MD) simulations numerically solve Newton's equations of motion of atoms in a molecular system to obtain information about its time-dependent properties, which leads to detailed information on the fluctuations and conformational changes of proteins and nucleic acids. An accurate force field can play a crucial role on the dynamical behavior of nucleic acids. All simulations were performed using the ff99 Cornell force field for RNA (Auffinger, P.; Westhof, E. Curr Opin Struct Biol 1998, 8, (2), 227-36) which has been shown to be a reliable and refined force field for nucleic acids, and the molecular dynamics software Amber 8.0 (Case, D. Aet al. AMBER 8, University of California San Francisco, 2004). In this research explicit and implicit solvation approaches were used to calculate salvation energies. The explicit solvent method (Auffinger, P. as above) produces the most accurate modeling of solvation effects and can provide crucial information on the direct interactions of water and ions with nucleic acids; however, it is slow and computationally expensive due to the presence of thousands of water molecules. Implicit solvation methods (Lee, M. S., Salsbury Jr., F. R., Brooks III, C. L. J. Chem. Phys. 2002, 116, 10606-10614) are based on the approximation of electrostatic solvation free energies assuming that the solvent is a uniform high-dielectric constant medium that responds to the partial charges of a low-dielectric constant solute. Implicit solvation methods permit simulations for longer computational times and larger molecules. The NMR solution structure of the RNAI/RNAII inverse kissing loop complex was subjected to explicit molecular dynamics simulations. The hexagonal RNA ring was subjected to implicit solvent molecular dynamics simulations.

Explicit solvent. The RNA Ii/RNA IIi complex was first neutralized with 38 Na+ ions. A water box containing 12565 molecules and additional 30 Na+ and 30 Cl− ions were added to represent 0.1 M solution. The electrostatic interactions were calculated by Particle Mesh Ewald summation (PME) (Essmann, U.; Perera, L.; Berkowitz, M. L.; Darder, T. A.; Lee, H.; Pedersen, L. G. J. Chem. Phys. 1995, 103, 8577) and the non-bonded interactions were truncated at 9 Å. The system was minimized constraining the solute then solvent, then heated to 300 K constraining the RNA then the solvent, and finally equilibrated by slowly releasing the constraints. SHAKE was applied to all hydrogen bonds in the system. Pressure was maintained at 1.0 Pa using Berendsen algorithm (Berendsen, H. J. C., Postma, J. P. M., van Gunsteren, W. F., DiNola, A., Haak, J. R. J. Chem. Phys. 1984, 81, 3684-3690), and a periodic boundary condition was imposed. A production simulation was performed for 13 ns with a 2 fs timestep.

Implicit solvent. Molecular dynamics simulations at 300 K constant temperature using the Generalized Bom (GB) implicit solvent approach as implemented in the SANDER module of Amber 8.032 were performed for nano-ring structure. The starting structure was subjected to minimization (10000 steps), followed by slow 20 kcal/mol constrained heating to 300 K over 200 ps time, and several consecutive MD equilibrations with declining constraints from 2 kcal/mol to 0.1 kcal/mol over a total 500 ps time period. The temperature was maintained at 300 K using a Berendsen thermostat (Essmann, U., as above). The monovalent salt concentration was set to 0.5 mol/L. The production simulations were performed for 8 ns using 1 fs time step.

The simulations were carried out on SGI-Altix and SGI-Origin computers using 8 processors. The analysis for all simulations was performed using the PTRAJ modules on the production simulations excluding the initial equilibration stage.

### INCORPORATION BY REFERENCE

The contents of all references, patents, pending patent applications and published patents, cited throughout this application are hereby expressly incorporated by reference.

### LITERATURE CITED

1. Jaeger, L.; Chworos, A. Curr Opin Struct Biol 2006, 16, (4), 531-43.
2. Yano, J.; Hirabayashi, K.; Nakagawa, S.; Yamaguchi, T.; Nogawa, M.; Kashimori, I.; Naito, H.; Kitagawa, H.; Ishiyama, K.; Ohgi, T.; Irimura, T. Clin Cancer Res 2004, 10, (22), 7721-6.
3. Flynn, M. A.; Casey, D. G.; Todryk, S. M.; Mahon, B. P. J Inflamm (Lond) 2004, 1, (1),
4. Schiffelers, R. M.; Ansari, A.; Xu, J.; Zhou, Q.; Tang, Q.; Storm, G.; Molema, G.; Lu, P. Y.; Scaria, P. V.; Woodle, M. C. Nucleic Acids Res 2004, 32, (19), e149.
5. Howard, K. A.; Rahbek, U. L.; Liu, X.; Damgaard, C. K.; Glud, S. Z.; Andersen, M. O.; Hovgaard, M. B.; Schmitz, A.; Nyengaard, J. R.; Besenbacher, F.; Kjems, J. Mol Ther 2006.
6. Shu, D.; Moll, W.-D.; Deng, Z.; Mao, C.; Guo, P. Nano Letters 2004, 4, (9), 1717-1723.
7. Guo, P. J Nanosci Nanotechnol 2005, 5, (12), 1964-82.
8. Khaled, A.; Guo, S.; Li, F.; Guo, P. Nano Lett 2005, 5, (9), 1797-808.
9. Guo, S.; Huang, F.; Guo, P. Gene Ther 2006, 13, (10), 814-20.
10. Jaeger, L.; Leontis, N. B. Angew Chem Int Ed Engl 2000, 39, (14), 2521-2524.
11. Jaeger, L.; Westhof, E.; Leontis, N. B. Nucleic Acids Res 2001, 29, (2), 455-63.
12. Hansma, H. G.; Oroudjev, E.; Baudrey, S.; Jaeger, L. J Microsc 2003, 212, (Pt 3), 273-9.
13. Nasalean, L.; Baudrey, S.; Leontis, N. B.; Jaeger, L. Nucleic Acids Res 2006, 34, (5), 1381-92.
14. Horiya, S.; Li, X.; Kawai, G.; Saito, R.; Katoh, A.; Kobayashi, K.; Harada, K. Nucleic Acids Res Suppl 2002, (2), 41-2.
15. Horiya, S.; Li, X.; Kawai, G.; Saito, R.; Katoh, A.; Kobayashi, K.; Harada, K. Chem Biol 2003, 10, (7), 645-54.
16. Li, X.; Horiya, S.; Harada, K. J Am Chem Soc 2006, 128, (12), 4035-40.
17. Chworos, A.; Severcan, I.; Koyfinan, A. Y.; Weinkam, P.; Oroudjev, E.; Hansma, H. G.; Jaeger, L. Science 2004, 306, (5704), 2068-72.
18. Bates, A. D.; Callen, B. P.; Cooper, J. M.; Cosstick, R.; Geary, C.; Glidle, A.; Jaeger, L.; Pearson, J. L.; Proupin-Perez, M.; Xu, C.; Cumming, D. R. Nano Lett 2006, 6, (3), 445-8.
19. Koyfinan, A. Y.; Braun, G.; Magonov, S.; Chworos, A.; Reich, N. O.; Jaeger, L. J Am Chem Soc 2005, 127, (34), 11886-7.
20. Chelyapov, N.; Brun, Y.; Gopalkrishnan, M.; Reishus, D.; Shaw, B.; Adleman, L. J Am Chem Soc 2004, 126, (43), 13924-5.
21. Mathieu, F.; Liao, S.; Kopatsch, J.; Wang, T.; Mao, C.; Seeman, N. C. Nano Lett 2005, 5, (4), 661-5.
22. Tsai, C. J.; Zheng, J.; Aleman, C.; Nussinov, R. Trends Biotechnol 2006, 24, (10), 449-54.
23. Lee, A. J.; Crothers, D. M. Structure 1998, 6, (8), 993-1005.
24. Tomizawa, J. J Mol Biol 1990, 212, (4), 695-708.
25. Eguchi, Y.; Tomizawa, J. J Mol Biol 1991, 220, (4), 831-42.
26. Dykxhoom, D. M.; Lieberman, J. Annu Rev Med 2005, 56, 401-23.
27. Izquierdo, M. Cancer Gene Ther 2005, 12, (3), 217-27.
28. Gregorian, R. S., Jr.; Crothers, D. M. J Mol Biol 1995, 248, (5), 968-84.
29. Zuker, M.; Mathews, D. H.; Turner, D. H., Algorithms and Thermodynamics for RNA Secondary Structure Prediction: A Practical Guide in RNA Biochemistry and Biotechnology. Kluwer Academic Publishers: 1999.
30. Shapiro, B. A.; Bengali, D.; Kasprzak, W.; Wu, J. C. J Mol Biol 2001, 312, (1), 27-44.
31. Wang, J. M., Cieplak, P., Kollman, P. A. J. Comp. Chem. 2000, 21 1049-1074.
32. Case, D. A.; Darden, T. A.; Cheatham III, T. E., Simmerling, C. L.,; Wang, J., Duke, R. E., Luo, R., Merz, K. M., Wang, B., pearlman, D. A.,Crowley, M.,; Brozell, S., Tsui, V., Gohlke, H., Mongan, J., Hornak, V., Cui, G., Beroza, P., Schafmeister, C., ; Caldwell, J. W.; Ross, W. S., Kollman, P. A. AMBER 8, University of California San Francisco, 2004.
33. Auffinger, P.; Westhof, E. Curr Opin Struct Biol 1998, 8, (2), 227-36.
34. Lee, M. S., Salsbury Jr., F. R., Brooks III, C. L. J. Chem. Phys. 2002, 116, 10606-10614.
35. Essmann, U.; Perera, L.; Berkowitz, M. L.; Darder, T. A.; Lee, H.; Pedersen, L. G. J. Chem. Phys. 1995, 103, 8577.
36. Berendsen, H. J. C., Postma, J. P. M., van Gunsteren, W. F., DiNola, A., Haak, J. R. J. Chem. Phys. 1984, 81, 3684-3690.

### Equivalents

Those skilled in the art will recognize, or be able to ascertain using no more than routine experimentation, many equivalents to the specific embodiments of the invention described herein. Such equivalents are intended to be encompassed by the following claims.

1. A polyvalent RNA nanoparticle comprising RNA motifs as building blocks.

2. The polyvalent RNA nanoparticle of claim 1, wherein the building blocks comprise a motif that allows for non-covalent assembly between 2 or more building blocks.

3. The polyvalent RNA nanoparticle of claim 1, wherein the RNA motifs are RNA I or RNA II motifs.

4-5. (canceled)

6. The polyvalent RNA nanoparticle of claim 1, wherein the RNA motifs are RNA I inverse (RNA Ii) or RNA II inverse (RNA IIi) motifs.

7. (canceled)

8. The polyvalent RNA nanoparticle of claim 1, wherein the polyvalent RNA nanoparticle is in the shape of a ring.

9. The polyvalent RNA nanoparticle of claim 8 wherein the ring comprises one or more building blocks.

10. The polyvalent RNA nanoparticle of claim 9, wherein the ring comprises 6 building blocks that form a hexameric ring.

11. The polyvalent RNA nanoparticle of claim 10, wherein there is a 120 degree angle at the loop-loop interactions at the corners of the hexameric ring.

12. The polyvalent RNA nanoparticle of claim 1, wherein the polyvalent RNA nanoparticle is in the shape of a square.

13-32. (canceled)

33. A drug delivery composition comprising the polyvalent RNA nanoparticle of claim 1, wherein the drug delivery composition can gain entry into a cell or tissue.

34. The drug delivery composition of claim 33, further comprising a second therapeutic agent.

35. The drug delivery composition of claim 34, wherein the second therapeutic agent is selected from the group consisting of: chemotherapeutic agents, cardiovascular drugs, respiratory drugs, sympathomimetic drugs, cholinomimetic drugs, adrenergic or adrenergic neuron blocking drugs, analgesics/antipyretics, anesthetics, antiasthmatics, antibiotics, antidepressants, antidiabetics, antifungals, antihypertensives, anti-inflammatories, antianxiety agents, immunosuppressive agents, immunomodulatory agents, antimigraine agents, sedatives/hypnotics, antianginal agents, antipsychotics, antimanic agents, antiarrhythmics, antiarthritic agents, antigout agents, anticoagulants, thrombolytic agents, antifibrinolytic agents, hemorheologic agents, antiplatelet agents, anticonvulsants, antiparkinson agents, antihistamines/antipruritics, agents useful for calcium regulation, antibacterials, antivirals, antimicrobials, anti-infectives, bronchodialators, hormones, hypoglycemic agents, hypolipidemic agents, proteins, peptides, nucleic acids, agents useful for erythropoiesis stimulation, antiulcer/antireflux agents, antinauseants/antiemetics and oil-soluble vitamins, or combinations thereof.

36. The drug delivery composition of claim 35, wherein the chemotherapeutic agent is selected from the group consisting of: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine, mechlorethamine oxide hydrochloride rethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, improsulfan, benzodepa, carboquone, triethylenemelamine, triethylenephosphoramide, triethylenethiophosphoramide, trimethylolomelamine, chlornaphazine, novembichin, phenesterine, trofosfamide, estermustine, chlorozotocin, gemzar, nimustine, ranimustine, dacarbazine, mannomustine, mitobronitol,aclacinomycins, actinomycin F(1), azaserine, bleomycin, carubicin, carzinophilin, chromomycin, daunorubicin, daunomycin, 6-diazo-5-oxo-1-norleucine, doxorubicin, olivomycin, plicamycin, porfiromycin, puromycin, tubercidin, zorubicin, denopterin, pteropterin, 6-mercaptopurine, ancitabine, 6-azauridine, carmofur, cytarabine, dideoxyuridine, enocitabine, pulmozyme, aceglatone, aldophosphamide glycoside, bestrabucil, defofamide, demecolcine, elfornithine, elliptinium acetate, etoglucid, flutamide, hydroxyurea, lentinan, phenamet, podophyllinic acid, 2-ethylhydrazide, razoxane, spirogermanium, tamoxifen, taxotere, tenuazonic acid, triaziquone, 2,2′,2″-trichlorotriethylamine, urethan, vinblastine, vincristine, vindesine and related agents. 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin IlIl derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase;. metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyidinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; taxel; taxel analogues; taxel derivatives; palauamine; paimitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin. Additional cancer therapeutics include monoclonal antibodies such as rituximab, trastuzumab and cetuximab.

37. A method of treating or preventing a disease or disorder, the method comprising administering to a subject in need thereof an effective amount of a polyvalent RNA nanoparticle or nanotube of claim 1.

38. The method of claim 37, wherein the disease or disorder is selected from the group consisting of: Adenoma, Ageing, AIDS, Alopecia, Alzheimer's disease, Anemia, Arthritis, Asthma, Atherosclerosis, Cancer, Cardiac conditions or disease, Diabetes mellitus, Foodborne illness, Hemophilia A-E, Herpes, Huntington's disease, Hypertension, Headache, Influenza, Multiple Sclerosis, Myasthenia gravis, Neoplasm, Obesity, Osteoarthritis, Pancreatitis, Parkinson's disease, Pelvic inflammatory disease, Peritonitis, Periodontal disease, Rheumatoid arthritis, Sepsis, Sickle-cell disease, Teratoma, Ulcerative colitis, Uveitis.

39. A method for making the polyvalent nanoparticle of claim 1, the method comprising:
overexpressing an RNA sequence comprising an RNA motif in a cell; and
allowing the RNA sequences to assemble into a polyvalent nanoparticle;
thereby making a polyvalent nanoparticle.

40. The method of claim 39, wherein the RNA motifs are RNA I or RNA II motifs.

41. The method of claim 40, wherein the motifs are kissing loops.

42. The method of claim 39, wherein the RNA motifs are RNA I inverse (RNA Ii) or RNA II inverse (RNA IIi) motifs.

43. The method of claim 42, wherein the RNAIi sequence comprises cggaugguucg (SEQ ID NO: 1) and the RNAIIi sequence comprises cgaaccauccg (SEQ ID NO: 2).

44. The method of claim 43, wherein the RNAIi and RNAIIi sequences are at opposite ends of a RNA nanoring building block.

45. The method of claim 44, wherein the RNA nanoring building block comprises the sequence: gcgc UAUCCAG gggaacggaugguucguuccc UUGGGUA gcgc CCCGUUCUCM ccgcaccgaaccauccggugcgg UUGGGMCGGG aa CUCUCUCUCUAGAGAGAGAG (SEQ ID NO: 3).

46. The method of claim 39, wherein the nanoparticle is at least 25 nm in size.
